(19) United States
(12) Patent Application Publication
(10) Pub. No.: US 2011/0264174 A1
(43) Pub. Date: Oct. 27, 2011

(54) Light Emmitting Apparatus

(75) Inventors: Andrew McNeill, Fife (GB); Jono Taylor, Bristol (GB)

(73) Assignee: AMBICARE HEALTH LIMITED, Edinburgh (GB)

(21) Appl. No.: 13/132,123

(22) PCT Filed: Dec. 1, 2009
(86) PCT No.: PCT/GB2009/051628

§ 371 Date: Jul. 13, 2011

(30) Foreign Application Priority Data
Dec. 1, 2008 (GB) 0821862.0

(51) Int. Cl. A61N 5/06

(52) U.S. Cl. 607/88

(57) ABSTRACT

There is herein described light emitting apparatus capable of adapting and/or conforming to a non-planar surface on a patient's body.

### FIELD OF THE INVENTION

The present invention relates to light emitting apparatus. More particularly, the present invention relates to light emitting apparatus capable of adapting and/or conforming to a non-planar surface on a patient's body.

### BACKGROUND OF THE INVENTION

Traditional light sources are typically rigid structures and are not flexible. Moreover, these types of traditional light sources contain electronics which further adds to their rigidity. Traditional light sources that do flex have been found to be prone to breaking (or disconnecting from electronics) when folded past a certain angle. Therefore, in applications where a flat light source is required to conform to a neighbouring surface, this lack of flex limits the degree of conformability.

Moreover, even though some prior art light sources exist these types of light sources only allow a certain amount of flexibility in one direction, but not in two. This is sufficient for illumination of small surfaces but not for larger surfaces that may have contours in many directions at different points. A light emitting surface that is highly flexible and can bend in different directions at different points on this surface is therefore required.

Light can be used to treat a wide variety of diseases. When light alone is used to treat a disease, the treatment is referred to as phototherapy. Light may be used in conjunction with a pharmaceutical in which case the treatment is called photodynamic therapy.

Phototherapy and photodynamic therapy can be used to treat a variety of skin and internal diseases. In photodynamic therapy, a light-sensitive therapeutic agent known as a photopharmaceutical is supplied externally or internally to an area of the body which is to be treated. That area is then exposed to light of a suitable frequency and intensity to activate the photopharmaceutical. A variety of photopharmaceutical agents are currently available. For example there are topical agents such as 5-aminolevulinic acid hydrochloride (Crawford Pharmaceuticals), methylaminolevulinic acid (Metfix (Trade Mark), Photocure). Often, the drug is applied in a non-active form that is metabolised to a light-sensitive photopharmaceutical.

In photodynamic therapy, the primary technique for supplying light to the photopharmaceutical is to project light of a suitable wavelength from standalone light sources such as lasers or filtered arc lamps. These sources are cumbersome and expensive, and are therefore only suitable for use in hospitals. This leads to inconvenience for the patient, and high cost for the treatment. High light irradiances are needed in order to treat an acceptable number of patients per day (for the treatment to be cost effective) and to avoid unduly inconveniencing the patient.

In order to achieve an effective photodynamic or phototherapy treatment, it is important that the area to be treated must be illuminated evenly. This ensures a consistent dose of light is administered across the treatments area. Other prior art light sources that are flexible consist of individual illuminators that are point sources. These point sources could be light emitting diodes embedded in a substrate that exhibits some degree of flexibility. The point illumination like nature of these light sources makes them intrinsically unsuited for homogenous illumination of treatment surfaces. The embedded nature of these light sources also means that as these light apparatus are folded or flexed, the illumination homogeneity is further reduced due to redirection and realignment of the individual point sources.

It is an object of at least one aspect of the present invention to obviate or mitigate at least one or more of the aforementioned problems.

It is a yet further object of at least one aspect of the present invention to provide light emitting apparatus comprising a light source wherein said medical apparatus is capable of adapting and/or conforming in a number of different directions.

### SUMMARY OF THE INVENTION

According to a first aspect of the present invention there is provided a light emitting apparatus for use in therapeutic and/or cosmetic treatment, the apparatus comprising:

at least two rigid substrates;

a flexible hinge connecting the at least two rigid substrates;

wherein each substrate contains at least one light source; and

a diffusing member;

wherein the light emitting apparatus is capable of adapting and/or conforming to a non-planar surface.

The apparatus may therefore be a medical light emitting apparatus which may be used for a variety of therapeutic and/or cosmetic treatments.

The rigid substrates connected by a flexible hinge with each substrate containing at least one light source may therefore be adaptable and/or conformable to and/or around a non-planar surface. In particular, this light apparatus may bend, flex and/or conform around a non-planar, substantially non-planar and/or curved surface.

The material forming the flexible hinge may be pliable and may bend, flex and/or conform. The flexible hinge may be an extended 2-dimensional planar structure in the form of, for example, a sheet-like structure. There may be at least one or a plurality of hinges connecting at least two or a plurality of rigid substrates. For example, a flexible material (e.g. a flexible polymeric material) such as a thermoplastic elastomer polymer may be used. This flexible material may continue around the periphery of rigid substrate and may act as a connecting hinge to any other adjacent rigid substrates; thereby creating a flexible tiled array of rigid substrates.

The material forming the flexible hinge may be rigid and/or may be flexible via, for example, a mechanical action. Each rigid substrate may be connected via a separate hinge. The material forming the flexible hinge and the apparatus itself may therefore flex in more than one direction at multiple points.

The thickness of the or each rigid substrate may be sufficient to provide support to the at least one light source and may also be thick enough to incorporate a diffusing member. Typically, each rigid substrate may have a thickness of about 0.01-15 mm, about 0.01-5 mm, about 0.01-2 mm or about 0.1-1 mm.

The rigid substrates connected by a flexible hinge, with each substrate containing at least one light source may therefore advantageously bend, flex and/or conform in different directions at different points on the surface of the flexible substrate. For example, rigid substrates connected by a flexible hinge, with each substrate containing at least one light source may be capable of bending, flexing and/or conforming in at least one or more, a plurality or multiple directions at the same or different points on the surface of the flexible substrate. Each rigid substrate may therefore flex in any direction. This is because any individual substrate may be or is surrounded by other substrates each of which provides a hinge to allow such flexing.

The rigid substrates may be attached and/or arranged in the form of tiles onto the material forming the flexible hinge. The rigid substrates may therefore be arranged in the form of tiles in a 2-dimensional or substantially 2-dimensional form on the flexible hinge. This is in contrast to the prior art which only has 1-dimensional arrays.

Typically, there may be at least two or more, or a plurality of rigid substrates connected via a flexible hinge to form a flexible tiled array. The light sources may be arranged in a regular or irregular pattern.

Light from the light source(s) may be easily distributed across a single rigid substrate in a homogenous manner; this may be achieved via a diffusing structure. It therefore follows that tiling of more than one such rigid substrate will create a much larger area in which light is distributed homogenously.

The light sources may be of any suitable shape and may, for example, be substantially circular, triangular, square, rectangular, pentagonal, hexagonal or any combination thereof.

The rigid substrates containing at least one light source may be attached to the flexible hinge using any suitable mechanical and/or adhesive means. For example, the light source(s) may be adhered using glue.

The substrates containing at least one light source may be attached to the flexible hinge in a manner that allows the resulting flexible apparatus to adapt and/or conform to a non-planar, substantially non-planar or curved surface. This may therefore allow all or substantially all of the light emitting apparatus to bend, flex and/or conform in multiple directions at different points.

Preferably, the light sources and diffusers may be located on the rigid substrate so that they do not touch one another as the flexible apparatus bends, flexes and/or conforms away from a planar position. Therefore, at least some or preferably all of the sides of the light sources do not touch when the flexible apparatus is substantially planar (i.e. flat) or bent, flexed and/or conformed.

The rigid substrates may have a bevelled profile so that each rigid substrate does not touch another as the flexible apparatus bends, flexes and/or conforms away from a planar position.

The sides of the rigid substrates may therefore be separated by a small distance of about 0.1-20 mm, about 0.1-10 mm, about 0.1-5 mm or about 1-5 mm.

The light emitting apparatus may have a light emitting surface area made up of a plurality of rigid substrates wherein each substrate contains at least one light source, a diffuser and an emitting surface of about 0.001-15 cm², about 0.01-5 cm², about 0.01-1 cm², about 0.01-0.5 cm² or about 0.01-0.5 cm². The rigid substrates may be of the same size or different. There may, for example, be about 1-10,000, about 1-1,000, about 1-1000 or about 1-10 small rigid substrates each joined by a flexible hinge to form a flexible apparatus. Alternatively, there may be more than 1, more than 10, more than 20, more than 50, more than 100 or more than 1,000 rigid substrates each joined by a flexible hinge to form a flexible apparatus. The total light emitting surface area made up from a plurality of rigid substrates each containing a light source, diffuser and light emitting surface may be about 0.01-500 cm², about 0.1-250 cm², about 0.1-100 cm², about 1-100 cm² or about 5-50 cm². Alternatively, the total light emitting surface area made up from a plurality rigid substrates each containing a light source, diffuser and light emitting surface may be greater than about 0.01 cm², greater than about 0.1 cm², greater than about 1 cm², greater than about 5 cm², greater than about 10 cm², greater than about 50 cm² or greater than about 100 cm².

The light emitting apparatus of the present invention may therefore provide light emitting apparatus which is capable of adapting and/or conforming to a non-planar, substantially non-planar or curved surface on, for example, any part of a human or mammal body such as on a foot, leg, torso, shoulder, arm, hand, finger, head or facial area of a patient. As the light emitting apparatus may be flexible this allows relatively large areas of a patient to be covered and treated as although individual substrate may be rigid or at least semi-rigid the light emitting apparatus as a whole may be flexible.

By allowing the light emitting apparatus to adapt and/or conform to a non-planar or curved surface allows the light emitting apparatus to be capable of providing even or substantially even illumination of a patient's skin, which is important for efficacious therapy. The present invention may therefore allow at least one, two or more, or a plurality of rigid or semi-rigid light sources located on the flexible substrate to adapt and/or conform to non-planar and/or curved surfaces on a patient.

The flexible apparatus may comprise a surface intended to make contact with a patient (i.e. a contact surface). This contact surface may be capable of bending, flexing and/or conforming around a non-planar, substantially non-planar or curved surface of, for example, a patient. Preferably, all or substantially all of the flexible apparatus is capable of making contact with a patient during use.

The flexible apparatus with the rigid substrates each containing a light source, diffuser and light emitting surface may be capable of bending, flexing and/or conforming away from a planar position in any direction by less than about 5 degrees, less than about 10 degrees, less than about 15 degrees, less than about 20 degrees, less than about 25 degrees, less than about 30 degrees, less than about 35 degrees, less than about 40 degrees, less than about 45 degrees, less than about 50 degrees, less than about 55 degrees, less than about 60 degrees, less than about 65 degrees, less than about 70 degrees, less than about 75 degrees, less than about 85 degrees or less than about 90 degrees. Alternatively, the flexible substrate with the light source(s) may be capable of bending, flexing and/or conforming away from a planar position in any direction by about 0-5 degrees, about 0-10 degrees, about 0-15 degrees, about 0-20 degrees, about 0-25 degrees, about 0-30 degrees, about 0-35 degrees, about 0-40 degrees, about 0-45 degrees, about 0-50 degrees, about 0-55 degrees, about 0-65 degrees, about 0-70 degrees, about 0-75 degrees, about 0-80 degrees, about 0-85 degrees or about 0-90 degrees.

The flexible apparatus with the rigid substrates each containing a light source, diffuser and light emitting surface may be capable of bending, flexing and/or conforming away from a planar position in any direction to a radius of curvature that is matched to the radius of curvature of any part of a human or mammal body such as on a foot, leg, torso, shoulder, arm, hand, finger, head or facial area of a patient.

The light emitting apparatus may comprise electronics for the operation of the apparatus and the at least one light source. The electronics may also be flexible and be capable of bending, flexing and/or conforming away from a planar position.

Each rigid substrate may also be designed to allow light from the at least one light source to exit from the light emitting apparatus and be used in medical applications such as therapeutic and/or cosmetic treatment. The light emitting apparatus may therefore be capable of distributing light over an area of a patient to be treated and preferably substantially evenly over an area of a patient to be treated.

The light source(s) may be located on top of a surface of the rigid substrate or alternatively may be located in suitably shaped apertures in the rigid substrate.

The light source(s) may be located at the side of each rigid substrate and light may be distributed over the surface of the rigid substrate via a diffusing member.

The light source(s) may be intrinsic area emitters such as an organic light emitting diode. In this case each rigid substrate will be an individual organic light emitting diode connected via a flexible hinge.

The flexible light emitting apparatus intended to make contact with a patient may have a surface area of about 0.1-500 cm², about 0.1-250 cm², about 0.1-100 cm², about 1-100 cm² or about 5-50 cm². Alternatively, the flexible light emitting apparatus intended to make contact with a patient may have a surface area of greater than about 0.01 cm², greater than about 0.1 cm², greater than about 1 cm², greater than about 5 cm², greater than about 10 cm², greater than about 50 cm² or greater than about 100 cm².

The flexible apparatus may also comprise an adhesive sheet around at least part or the entire periphery of the flexible substrate. The adhesive sheet may provide an adhesive surface for attaching the light emitting apparatus to a patient.

The flexible apparatus may also comprise a transparent adhesive sheet that sits between the flexible apparatus and the skin. The adhesive sheet may provide an adhesive surface for attaching the light emitting apparatus to a patient.

The apparatus may also comprise a drug and/or chemical source capable of delivering drugs and/or chemicals to the area of the patient to be treated. The apparatus of the present invention may therefore be capable of performing a therapeutic and/or cosmetic treatment.

The present invention may therefore relate to light emitting apparatus capable of performing a therapeutic and/or cosmetic treatment on a human or animal patient using photodynamic therapy or phototherapy. In some situations the treatment may be said to be of therapeutic nature (e.g. skin cancer, serious acne) whereas in other situations the treatment may be said to be of cosmetic nature (e.g. minor amounts of acne, anti-aging treatments such as treatment of wrinkling).

The light source may be any suitable light source that may emit light over a desired wavelength. The light source may be provided as a light emitting layer. Typically, the light source may operate within a range of about 300-3000 nm, about 300-1500 nm, about 300-800 nm or about 370-700 nm. In particular embodiments the light source may operate in the visible region of the electromagnetic spectrum. Alternatively, the light source may operate in the ultraviolet or infrared wavelength regions of the electromagnetic spectrum.

The light sources may emit light substantially continuously over a pre-set period of time or may emit light discontinuously such as in a pulsed manner. In particular embodiments, the light from the light source may be pulsed with a period of at least about 10 ms, at least about 100 ms, at least about 1 s, at least about 10 s, at least about 10 ms, at least about 100 s, at least about 1,000 s or at least about 10,000 s.

In particular embodiments, the light source may, for example, be any suitable form of diode such as organic light-emitting diode or an inorganic light-emitting diode incorporated into, for example, a diffuser. Alternatively, the light source may be a fluorescent light source such as a fluorescent lamp. The fluorescent light source may be compact in shape. For example, this includes technology developed from back-lit display technology, LEDs and waveguides and diffusers.

The at least one light source may have an optical power density of about 0.1-500 mW/cm², about 1-200 mW/cm² or about 5-50 mW/cm². The at least one light source during operation may operate at a substantially constant power or alternatively may be varied over a range of powers.

The apparatus may comprise a photochemical and/or a photopharmaceutical preparation in a drug and/or chemical source in the form of, for example, a layer.

The photochemical and/or photopharmaceutical preparation may be delivered to the area of the patient to be treated at appropriate pre-set times and/or may be controlled by a control unit. The photochemical and/or a photopharmaceutical preparation may be present in the form of, for example, a gel, ointment, cream or gauze soaked in a photodynamic therapy solution. Alternatively, or in addition the light emitting apparatus may be provided with a thin film impregnated with a photochemical and/or photopharmaceutical preparation. The photochemical and/or a photopharmaceutical preparation may comprise a drug and/or antiseptic capable of treating a patient which may be applied to a person in need thereof. Typically, the photochemical and/or photopharmaceutical may be transparent or substantially transparent or may become transparent or substantially transparent during use and emission of light.

In embodiments where the photochemical and/or photopharmaceutical may be transparent or translucent to the wavelength of the irradiating light, the resulting device may be readily applied without a separate step of applying a photochemical and/or a photopharmaceutical to a patient. In particular embodiments, the photochemical and/or photopharmaceutical may be covered with a peelable release medium. The photochemical and/or photopharmaceutical preparation may comprise an inactive compound which may be metabolised in vivo to an active compound. During use a suitable amount of the photochemical and/or photopharmaceutical preparation may be applied to an area of a patient to be treated.

Typical drugs and/or chemicals used in the present invention include but are not limited to precursor drugs such as ALA or Metfix or other photodynamic therapy agents.

In use, the drug and/or chemical layer may be placed over the top of the area of the patient to be treated. To facilitate treatment an electrolyte solution such as sodium chloride solution may be placed on the skin to improve the electrical contact.

A power source may be supplied to power the light source and/or the electronics on the flexible substrate. The power source may be small and compact and may be integrated into the whole apparatus therefore making the apparatus suitable for ambulatory treatment. The apparatus of the present invention may therefore be wearable and may be attached to a foot, leg, torso, shoulder, arm, hand, head or facial area of a patient.

The light emitting apparatus may comprise attachment means for attaching the device to a body part(s) of a human or animal. For example, the apparatus may comprise mechanical and/or adhesive means for attaching the device to a body part of a human or animal. In particular embodiments, the light emitting apparatus may therefore comprise a strap arrangement which may optionally comprise a fastening means and/or Velcro (Trade Mark) and/or an adhesive surface for attaching the apparatus to the patient.

The light emitting apparatus may be lightweight and portable. In particular embodiments the apparatus may be a totally self-contained portable unit and may comprise a self-contained power supply. The power supply may operate electronics in the device and the light source. The light emitting apparatus may be sufficiently portable to enable ambulatory treatment therefore allowing treatment during which a patient may move around. Treatment may therefore occur at home or at work and may be removed by a patient when necessary. This provides lower treatment costs as this avoids out-patient or in-patient stays in hospital. This provides the significant advantage that lower light levels may be used since exposure can occur for a longer period of time. This overcomes the problem of pain induced in some patients by high irradiances from conventional sources used in hospitals. Moreover, lower irradiance over a longer period of time may be more effective in photoinduced therapy as it gives more time for oxygen to diffuse to the region to be treated, and reduces photobleaching of the photopharmaceutical.

The at least one light source may also comprise a substrate layer such as a transparent or at least a substantially transparent substrate layer. Alternatively, the at least light source may comprise a translucent or at least a substantially translucent substrate layer. The substrate layer may function as a support layer for the light source and may allow the light to penetrate there through. The substrate layer may also function as a barrier layer too and may be selected to prevent oxygen and/or moisture from penetrating the light source. The substrate layer may be made from or comprise any one of or combination of suitable materials such as a glass, plastics or polymer. Additional layers may also be present.

The apparatus according to the present invention may be used in a range of phototherapies and photodynamic therapies. For example, the apparatus according to the present invention may be used in the treatment of cancer (e.g. skin cancer), acne, wrinkles, wound-healing, anti-aging and post-skin laser treatments such as found in cosmetic applications. By wound is meant any form of open or closed wound. Open wounds include but are not limited to: incisions or incised wounds; lacerations; abrasions; puncture wounds; penetration wounds; gunshot wounds; and ulcers (including diabetic derived ulcers). Closed wounds include but are not limited to: contusions; hemaomas; and crushing injuries.

According to a second aspect of the present invention there is provided a method of performing a therapeutic and/or cosmetic treatment, the method comprising:

providing at least two rigid substrates;

providing a flexible hinge capable of connecting the at least two rigid sources;

wherein each substrate contains at least one light source; and

a diffusing member

wherein the light emitting apparatus is a capable of adapting and/or conforming to a non-planar surface.

The method may be performed by an apparatus as defined in the first aspect.

According to a third aspect of the present invention there is provided use of an apparatus according to the first aspect in a medical treatment.

Typically, the medical treatment may be a photodynamic therapy or phototherapy.

The present invention may therefore relate to the treatment of cancer (e.g. skin cancer), acne, wrinkles, wound-healing, anti-aging and post-skin laser treatments (e.g. cosmetic applications).

According to a fourth aspect of the present invention there is provided a method of medical treatment using of an apparatus according to the first aspect.

The medical treatment may be that of photodynamic therapy or phototherapy.

Moreover, the treatment may be that of the treatment of cancer (e.g. skin cancer), acne, wrinkles, wound-healing, anti-aging and post-skin laser treatments (e.g. cosmetic applications).

### BRIEF DESCRIPTION OF THE DRAWINGS

Embodiments of the present invention will now be described, by way of example only, with reference to the accompanying drawings in which:

FIG. 1 is a representation of a light emitting apparatus according to an embodiment of the present invention.

### BRIEF DESCRIPTION

The present invention therefore relates to an apparatus capable of performing a therapeutic and/or cosmetic treatment on a human or animal patient. The apparatus may be used for any type of medical treatment and, for example, may be used in phototherapy or photodynamic therapy.

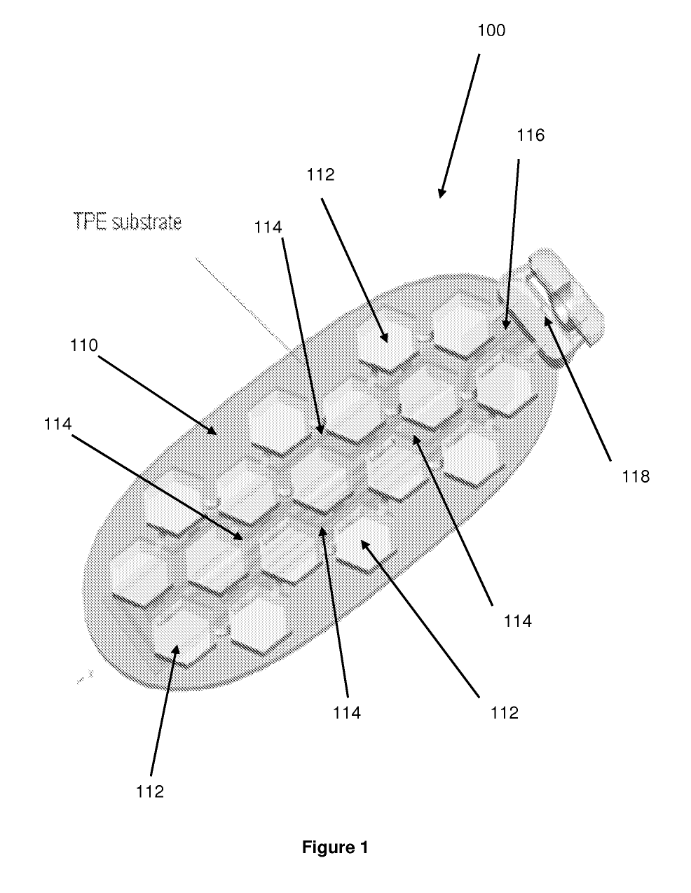

FIG. 1 is a representation of a light emitting apparatus according to the present invention, generally designated 100. The light emitting apparatus 100 comprises a number of rigid substrates 112. in a regular pattern.

As shown in FIG. 1, there are a series of flexible hinges 114 between the rigid substrates 112 and these areas may bend, flex and/or conform and thereby allow the whole light emitting apparatus 100 to bend, flex and/or conform in multiple directions at different points on the light emitting apparatus 100.

Each rigid substrate contains at least one light source and a diffusing member.

The light emitting apparatus 100 also comprises electric cables 116 for the operation of the light sources within the rigid substrates 112. The electric cables 116 enter the flexible apparatus 110 via entrance 118.

Whilst specific embodiments of the present invention have been described above, it will be appreciated that departures from the described embodiments may still fall within the scope of the present invention. For example, any suitable type of flexible substrate may be used. Furthermore, any suitable type of light source may be used.

1-41. (canceled)

42. A light emitting apparatus for use in therapeutic and/or cosmetic treatment, the apparatus comprising:
at least two rigid substrates;
a flexible hinge connecting the at least two rigid substrates;
wherein each substrate contains at least one light source; and
a diffusing member;
wherein the light emitting apparatus is a capable of adapting and/or conforming to a non-planar surface.

43. The apparatus of claim 42, wherein the flexible hinge is an extended 2-dimensional planar structure in the form of, for example, a sheet-like structure, wherein material forming the flexible hinge is pliable and is capable of bending, flexing and/or conforming, and wherein the light emitting apparatus comprises at least one or a plurality of hinges connecting at least two or a plurality of rigid substrates.

44. The apparatus of claim 43, wherein material forming the flexible hinge is made from a flexible polymeric material.

45. The apparatus of claim 42, wherein the rigid substrates are attached and/or arranged in the form of tiles onto material forming the flexible hinge in a 2-dimensional form, wherein each rigid substrate is therefore capable of flexing in any direction, and wherein a flexible material continues around a periphery of at least one of the rigid substrates and acts as a connecting hinge to any other adjacent rigid substrates, thereby creating a flexible tiled array of rigid substrates.

46. The apparatus of claim 42, wherein there is a plurality of rigid substrates each connected via a separate hinge; and wherein the thickness of the or each rigid substrate is sufficient to provide support to the at least one light source and is also thick enough to incorporate a diffusing member.

47. The apparatus of claim 42, wherein at least two rigid substrates have a thickness of about 0.01-15 mm, about 0.01-5 mm, about 0.01-2 mm or about 0.1-1 mm, the at least two rigid substrates are connected by a flexible hinge, with each substrate containing at least one light source allowing the light emitting apparatus to bend, flex and/or conform in different directions at different points on the surface of the flexible substrate; and the at least two rigid substrates are connected by a flexible hinge, with each substrate containing at least one light source, the light emitting apparatus being capable of bending, flexing and/or conforming in at least one or more, a plurality or multiple directions at the same or different points on the surface of the flexible substrate.

48. The apparatus of claim 42, wherein there is at least two or more, or a plurality of rigid substrates connected via flexible hinge to form a flexible tiled array.

49. The apparatus of claim 42, wherein light from the at least one light source is capable of being distributed across a single rigid substrate in a homogenous manner using a diffusing structure, wherein the light sources are substantially circular, triangular, square, rectangular, pentagonal, hexagonal or any combination thereof, and wherein the rigid substrates containing at least one light source are attached to the flexible hinge using any suitable mechanical and/or adhesive means

50. The apparatus of claim 42, wherein the light sources and diffusers are located on the rigid substrate so that they do not touch one another as the flexible apparatus bends, flexes and/or conforms away from a planar position; the rigid substrates are separated by a small distance of about 0.1-20 mm, about 0.1-10 mm, about 0.1-5 mm or about 1-5 mm; the light emitting apparatus has a light emitting surface area made up of a plurality of rigid substrates wherein each substrate contains at least one light source, a diffuser and an emitting surface of about 0.001-15 cm² , about 0.01-5 cm², about 0.01-1 cm², about 0.01-0.5 cm² or about 0.01-0.5 cm²; there is about 1-10,000, about 1-1,000, about 1-1000 or about 1-10 small rigid substrates each joined by a flexible hinge to form a flexible apparatus; and wherein the total light emitting surface area made up from a plurality of rigid substrates each containing a light source, diffuser and light emitting surface is about 0.01-500 cm², about 0.1-250 cm², about 0.1-100 cm², about 1-100 cm² or about 5-50 cm².

51. The apparatus of claim 42, wherein the light emitting apparatus is capable of adapting and/or conforming to a non-planar, substantially non-planar or curved surface on, for example, any part of a human or mammal body such as on a foot, leg, torso, shoulder, arm, hand, finger, head or facial area of a patient; wherein the flexible apparatus with the rigid substrates each containing a light source, a diffuser and light emitting surface is capable of bending, flexing and/or conforming away from a planar position in any direction by less than about 5 degrees, less than about 10 degrees, less than about 15 degrees, less than about 20 degrees, less than about 25 degrees, less than about 30 degrees, less than about 35 degrees, less than about 40 degrees, less than about 45 degrees, less than about 50 degrees, less than about 55 degrees, less than about 60 degrees, less than about 65 degrees, less than about 70 degrees, less than about 75 degrees, less than about 85 degrees or less than about 90 degrees; or wherein the flexible substrate with the light source(s) are capable of bending, flexing and/or conforming away from a planar position in any direction by about 0-5 degrees, about 0-10 degrees, about 0-15 degrees, about 0-20 degrees, about 0-25 degrees, about 0-30 degrees, about 0-35 degrees, 0-40 degrees, about 0-45 degrees, about 0-50 degrees, about 0-55 degrees, about 0-65 degrees, about 0-70 degrees, about 0-75 degrees, about 0-80 degrees, about 0-85 degrees, or about 0-90 degrees.

52. The apparatus of claim 42, wherein the light emitting apparatus with the rigid substrates each containing a light source, diffuser and light emitting surface are capable of bending, flexing and/or conforming away from a planar position in any direction to a radius of curvature that is matched to the radius of any part of a human or mammal body such as on a foot, leg, torso, shoulder, arm, hand, finger, head or facial area of a patient; wherein the light emitting apparatus comprises electronics for the operation of the apparatus and the at least one light source and the electronics are also flexible and are capable of bending, flexing and/or conforming away from planar position, and wherein the light source(s) are located at the side of each rigid substrate and light is capable of being distributed over the surface of the rigid substrate via a diffusing member.

53. The apparatus of claim 42, wherein the light source(s) are intrinsic area emitters including that of an organic light emitting diode and each rigid substrate is an individual organic light emitting diode connected via a flexible hinge.

54. The apparatus of claim 42, wherein the light emitting apparatus also comprise an adhesive sheet around at least part of the entire periphery of the flexible substrate, or wherein the light emitting apparatus comprises a transparent adhesive sheet that sits between the flexible apparatus and the skin.

55. The apparatus of any preceding claim, wherein light emitting apparatus comprises a drug and/or chemical source capable of delivering drugs and/or chemicals to an area of a patient to be treated, wherein the treatment is for skin cancer, serious acne, minor amounts of acne or anti-aging treatments including treatment of wrinkling, and wherein the light source(s) is capable of operating within a range of about 300-3000 nm, about 300-1500 nm, about 300-800 nm or about 370-700 nm.
